(19) United States
(12) Patent Application Publication
(10) Pub. No.: US 2026/0041428 A1
(43) Pub. Date: Feb. 12, 2026

(54) Implantable Cover Device for an Anastomosis Surgical Procedure and Related Surgical Methods

(71) Applicant: Evergreen Surgical Solutions, LLC, Indiana, PA (US)

(72) Inventor: Andrew Billon, Indiana, PA (US)

(21) Appl. No.: 19/100,145

(22) PCT Filed: Aug. 1, 2023
(86) PCT No.: PCT/US2023/029186

§ 371 Date: Jan. 31, 2025

Related U.S. Application Data
(60) Provisional application No. 63/394,120, filed on Aug. 1, 2022.

(51) Int. Cl. A61B 17/11; A61B 17/115

(52) U.S. Cl. CPC A61B 17/1114; A61B 17/115; A61B 2017/1132

(57) ABSTRACT

An implantable surgical cover device configured to be implanted over tissue of a patient at a surgical site during a surgical stapling procedure includes at least one sheet having at least one layer of amnion. The at least one sheet includes a first surface, a second surface, and a peripheral edge extending between the first surface and the second surface. The cover device also includes at least one annular opening extending through the at least one sheet provided to guide a user for proper positioning of the surgical stapler during the surgical stapling procedure. Surgical methods for anastomosis surgical procedures that include positioning a cover device or sheet including amnion at a surgical site are also described.

### CROSS REFERENCE TO RELATED APPLICATIONS

This application is the United States national phase of International Application No. PCT/US23/29186, filed Aug. 1, 2023, and claims the benefit of U.S. Provisional Patent Application No. 63/394,120, filed Aug. 1, 2022, entitled “Enhanced Anastomosis Method and System,” the disclosures of which are hereby incorporated by reference in their entireties.

### BACKGROUND OF THE INVENTION

Gastrointestinal (GI) or intestinal anastomosis (e.g., bowl anastomosis) is a common surgical technique used following damage or resection of GI sections, such as sections of the small or large intestines, or attachment of one portion of the GI tract to another, such as a sigmoid to rectal anastomosis. Two common surgical methods are side-to-side anastomosis (SSA) and end-to-end anastomosis (EEA). In both surgical methods, a connection is formed between a lumen of an open end of the GI tract and a lumen of another end of the GI tract.

Anastomosis procedures are often performed using a surgical stapler for deploying multiple staples for connecting a first tissue section to a second tissue section during the anastomosis procedure. Many different designs of surgical staplers are currently available including circular surgical staplers and staplers including elongated legs that provide linear or elongated staple regions in tissue. Surgical staplers can be manually operated by a surgeon and/or controlled by robotic-assisted surgery systems.

However, currently available surgical stapler devices and surgical methods are susceptible to complications and failures, which can include leakage, inflammation, and rupture. In particular, anastomotic leaks, which have an incidence of up to 20%, can lead to significant morbidity, such as peritonitis, abscess formation, sepsis and death. See Sripathi S, Khan M I, Patel N, Meda R T, Nuguru S P, Rachakonda S. Factors contributing to anastomotic leakage following colorectal surgery: Why, when and who leaks? Cureus. 2022 October; 10: e29964.

There is a need in the art for medical devices and surgical methods that address these complications in order to provide more secure connections, without leaks, between tissue sections in anastomosis surgical procedures. The surgical devices and methods disclosed herein are provided to address these complications.

### SUMMARY OF THE INVENTION

According to an aspect of the present disclosure, an implantable surgical cover device configured to be implanted over tissue of a patient at a surgical site during a surgical stapling procedure includes at least one sheet having at least one layer of amnion. The at least one sheet includes a first surface, a second surface, and a peripheral edge extending between the first surface and the second surface. The cover device also includes at least one annular opening extending through the at least one sheet provided to guide a user for proper positioning of the surgical stapler during the surgical procedure.

According to another aspect of the present disclosure, a surgical method includes a step of positioning at least one sheet including amnion between a first tubular section of patient tissue and a second tubular section of patient tissue. The method further includes a step of connecting the first tubular section to the second tubular section by at least one of stapling or sutures, such that at least some staples or sutures pass through the at least one sheet, thereby connecting the first tubular section to the second tubular section, with the at least one sheet including amnion between the first tubular section and the second tubular section.

According to another aspect of the present disclosure, a surgical method for forming a side-by-side anastomosis includes a step of positioning at least one sheet having amnion between a side surface of a first tubular section of patient tissue and a side surface of a second tubular section of patient tissue. The method further includes a step of inserting an anvil leg of a surgical stapler through an open end of one of the first tubular section or the second tubular section and inserting a staple leg of the surgical stapler through an open end of the other of the first tubular section or the second tubular section. The method further includes a step of connecting the first tubular section to the second tubular section by activating the stapler causing staples to pass from the staple portion, through the side surfaces of the first tubular section and the second tubular section, and through the at least one sheet. The method further includes positioning another at least one sheet comprising amnion over the open end of the first tubular section and the second tubular section; and forming a staple line passing through the side of the first tubular section, the side of the second tubular section, and the another at least one sheet. The staple line seals the open end of the first tubular section and the open end of the second tubular section forming a continuous lumen extending from the first tubular section to the second tubular section.

Other non-limiting examples of the present disclosure are set forth in the following illustrative numbered clauses:

Clause 1: An implantable surgical cover device configured to be implanted over tissue of a patient at a surgical site during a surgical stapling procedure, the cover device comprising: at least one sheet comprising at least one layer of amnion, wherein the at least one sheet comprises a first surface, a second surface, and a peripheral edge extending between the first surface and the second surface; and at least one opening extending through the at least one sheet provided to guide a user for proper positioning of a surgical stapler during the surgical stapling procedure, and wherein the opening is optionally an annular opening.

Clause 2: The cover device of clause 1, wherein the at least one layer of amnion comprises a dried sheet of chorion-free amnion.

Clause 3: The cover device of clause 1 or clause 2, wherein the at least one sheet comprises a one ply, two ply, three ply, or four ply amnion sheet.

Clause 4: The cover device of any of clauses 1-3, wherein the at least one sheet is a dried sheet comprising at least two layers of chorion-free amnion.

Clause 5: The cover device of any of clauses 1-4, wherein the at least one sheet is a square shape with an edge of from about 4 cm to about 8 cm in length, wherein the at least one annular opening has a diameter of from about 5 mm to about 20 mm, and wherein the opening is optionally an annular opening.

Clause 6: The cover device of any of clauses 1-5, wherein the at least one opening is sized to permit an anvil portion of a circular surgical stapler to pass through the at least one opening, and wherein the opening is optionally an annular opening.

Clause 7: The cover device of clause 1, wherein the at least one sheet is rectangular and the at least one opening is offset from a center of the at least one sheet by at least 5 mm in at least one direction, and wherein the opening is optionally an annular opening.

Clause 8: A surgical method comprising: positioning at least one sheet comprising amnion between a first tubular section of patient tissue and a second tubular section of the patient tissue; and connecting the first tubular section to the second tubular section by at least one of stapling or sutures, such that at least some staples or sutures pass through the at least one sheet, thereby connecting the first tubular section to the second tubular section, with the at least one sheet comprising amnion between the first tubular section and the second tubular section.

Clause 9: The method of clause 8, wherein the first tubular section defines a first lumen and the second tubular section defines a second lumen, and wherein connecting the first tubular section to the second tubular section places the first lumen in fluid communication with the second lumen.

Clause 10: The method of clause 9, wherein the at least one sheet comprises an opening extending through surfaces of the at least one sheet, wherein positioning the at least one sheet comprises positioning the at least one sheet over the first tubular section such that the opening is aligned with the first lumen, and wherein the opening is optionally an annular opening.

Clause 11: The method of clause 10, wherein connecting the first tubular section to the second tubular section comprises: positioning an anvil portion of a circular surgical stapler in the first lumen of the first tubular section, such that a stem of the anvil portion extends through the opening, e.g., annular opening, of the at least one sheet; positioning a staple portion of the circular surgical stapler in the second lumen of the second tubular section; tightening the circular surgical stapler, thereby causing the staple portion to move towards the anvil portion; and the first tubular section to move towards the second tubular section and activating the circular surgical stapler to project a plurality of staples from the staple portion through the first tubular section, the at least one sheet, and the second tubular section, thereby connecting the first tubular section to the second tubular section.

Clause 12: The method of clause 11, wherein the circular surgical stapler comprises a gastrointestinal stapler.

Clause 13: The method of clause 12, wherein the opening comprises a diameter that is slightly larger than a diameter of the anvil portion, thereby allowing the anvil portion to pass through the opening without contacting the at least one sheet, and wherein the opening is optionally an annular opening.

Clause 14: The method of any of clauses 8-13, wherein a surgical procedure of the surgical method is an end-to-end anastomosis in which an end of the first tubular section is connected to an end of the second tubular section.

Clause 15: The method of clauses 8-14, wherein a surgical procedure of the surgical method is a side-to-end anastomosis, in which an end of the first tubular section is connected over an opening in a sidewall of the second tubular section.

Clause 16: The method of clauses 8-15, wherein a surgical procedure of the surgical method is a side-to-side anastomosis.

Clause 17: The method of clause 16, wherein positioning the at least one sheet between the first tubular section and the second tubular section comprises positioning a first at least one sheet between a side surface of the first tubular section and a side surface of the second tubular section.

Clause 18: The method of clause 17, wherein connecting the first tubular section to the second tubular section comprises stapling the side surface of the first tubular section to the side surface of the second tubular section through the first at least one sheet, the method further comprising forming an incision through the side surface of the first tubular section, the first at least one sheet, and the side surface of the second tubular section, thereby forming a passage between a lumen defined by the first tubular section and a lumen of the second tubular section.

Clause 19: The method of clause 17 or clause 18, further comprising: positioning a second at least one sheet comprising amnion over open ends of the first tubular section and the second tubular section; and forming a staple line through the first tubular section and the second tubular section and the second at least one sheet comprising amnion, thereby sealing the open ends of the first tubular section and second tubular section and forming an enclosed lumen defined by the first tubular section and the second tubular section.

Clause 20: The method of clauses 8-19, wherein a surgical procedure of the surgical method is a robotic-assisted surgical procedure, and wherein connecting the first tubular section to the second tubular section comprises automatically stapling the first tubular section to the second tubular section using a surgical robot system comprising at least one arm that moves and actuates a surgical stapler.

Clause 21: The method of clauses 8-20, wherein a surgical procedure of the surgical method comprises an anastomosis procedure, and wherein the first tubular section and the second tubular section comprise sections of a gastrointestinal (GI) tract of a patient.

Clause 22: The method of clause 21, wherein the sections of the GI tract comprise at least one of a bowel or intestine of the patient.

Clause 23: A surgical method for forming a side-by-side anastomosis, comprising: positioning at least one sheet comprising amnion between a side surface of a first tubular section of patient tissue and a side surface of a second tubular section of the patient tissue; inserting an anvil leg of a surgical stapler through an open end of one of the first tubular section or the second tubular section and inserting a staple leg of the surgical stapler through an open end of the other of the first tubular section or the second tubular section; connecting the first tubular section to the second tubular section by activating the surgical stapler causing staples to pass from the staple leg, through the side surfaces of the first tubular section and the second tubular section, and through the at least one sheet; positioning another at least one sheet comprising amnion over the open end of the first tubular section and the second tubular section; and forming a staple line passing through the side surface of the first tubular section, the side surface of the second tubular section, and the another at least one sheet, wherein the staple line seals the open end of the first tubular section and the open end of the second tubular section forming a continuous lumen extending from the first tubular section to the second tubular section.

Clause 24: The method of clause 23, wherein a surgical procedure of the surgical method comprises an anastomosis procedure, and wherein the first tubular section and the second tubular section comprise sections of a gastrointestinal (GI) tract of a patient.

Clause 25: The method of clause 24, wherein the sections of the GI tract comprise at least one of a bowel or intestine of the patient.

Clause 26: The method of any of clauses 23-25, further comprising forming an incision through the side surface of the first tubular section, the at least one sheet, and the side surface of the second tubular section, thereby forming the continuous lumen defined by the first tubular section and the second tubular section.

Clause 27: The method of any of clauses 23-26, wherein a surgical procedure of the surgical method is a robotic-assisted surgical procedure, and wherein connecting the first tubular section to the second tubular section by activating the surgical stapler comprises automatically stapling the first tubular section to the second tubular section using a surgical robot system comprising at least one arm that moves and actuates the surgical stapler.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1A is a schematic drawing of an implantable cover device for positioning on a surgical site, according to an aspect of the present disclosure;

FIG. 1B is a schematic drawing of another example of an implantable cover device for positioning on a surgical site, according to an aspect of the present disclosure;

FIGS. 2A-2D are schematic drawings showing steps of a surgical method for forming a side-to-side anastomosis, according to an aspect of the present disclosure;

FIG. 2E is a schematic drawing showing tubular sections connected together by a side-to-side anastomosis surgical procedure, according to an aspect of the present disclosure;

FIGS. 3A-3E are schematic drawings showing steps of a surgical method for forming an end-to-end anastomosis, according to an aspect of the present disclosure;

FIG. 3F is a schematic drawing showing tubular sections connected together by a completed end-to-end anastomosis procedure, according to an aspect of the present disclosure;

FIG. 4 is a flow chart showing steps of a method for a side-to-side anastomosis surgical procedure, according to an aspect of the present disclosure; and

FIG. 5 is a flow chart showing steps of a method for an end-to-end anastomosis surgical procedure, according to an aspect of the present disclosure.

### DETAILED DESCRIPTION OF THE INVENTION

As used herein, the singular form of “a”, “an”, and “the” include plural referents unless the context clearly states otherwise.

As used herein, the terms “right”, “left”, “top”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

As used herein, the “patient” can be any species of the human or animal kingdom having a gastrointestinal tract including, for example, intestines, colon, and/or rectum. Non-limiting examples of patients include mammal(s), such as human(s) and/or non-mammalian animal(s). Non-limiting examples of mammal(s) include primate(s) and/or non-primate(s). Primate(s) include human(s) and non-human primate(s), including but not limited to male(s), female(s), adult(s) and children. Non-limiting examples of non-human primate(s) include monkey(s) and/or ape(s), for example chimpanzee(s). Non-limiting examples of non-primate(s) include cattle (such as cow(s), bull(s) and/or calve(s), pig(s), camel(s), llama(s), alpaca(s), horse(s), donkey(s), goat(s), rabbit(s), sheep, hamster(s), guinea pig(s), cat(s), dog(s), rat(s), mice, lion(s), whale(s), and/or dolphin(s). Non-limiting examples of non-mammalian animal(s) include bird(s) (e.g., duck(s) or geese), reptile(s) (e.g., lizard(s), snake(s), or alligator(s)), amphibian(s) (e.g., frog(s)), and/or fish. In some examples, the animals can be zoological animals, human pets and/or wild animals.

For the purposes of this specification, unless otherwise indicated, all numbers expressing quantities of ingredients, dimensions, physical characteristics, and so forth used in the specification and claims are to be understood as being modified in all instances by the term “about.” Unless indicated to the contrary, the numerical parameters set forth in the following specification and attached claims are approximations that can vary depending upon the desired properties sought to be obtained by the present invention. As used herein, the term “about” means “close to” and that variation from the exact value that follows the term is within amounts that a person of skill in the art would understand to be reasonable. For example, when the term “about” is used with respect to a numerical value, the value may vary within a reasonable range, such as within +/−10%, +/−5%, or +/−1% of the stated value. Notwithstanding that the numerical ranges and parameters setting forth the broad scope of the invention are approximations, the numerical values set forth in the specific examples are reported as precisely as possible. Any measured numerical value, however, may inherently contain certain errors resulting from the standard deviation found in their respective testing measurements. Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

With reference to the figures, the present disclosure is directed to medical devices, such as a substrate, patch, bandage, or cover device 10, configured to be implanted at a surgical site and to surgical methods for positioning and fixation of such devices 10. The medical devices and surgical methods disclosed herein are intended for use in surgical procedures, particularly anastomosis surgical procedures, namely formation of an end-to-end, end-to-side, and/or side-to-side anastomosis between tubular structures (e.g., a bowel or intestine) of a patient's GI tract.

The medical devices and methods of the present disclosure use sheet(s) of protective material, such as sheets or layers comprising amnion layer(s) and/or an amnion scaffold derived from placenta, for improving safety of anastomosis surgical procedures, reducing leakage, bleeding, inflammation, and rupture. In some examples, the protective material of the medical device (e.g., the substrate or cover device 10) is a single layer or multi-layer (e.g., two ply, three ply, or four ply) sheet comprising amnion. In some examples, the protective material or sheet comprises amnion that is free from chorion. A sheet that is entirely amnion and/or is chorion-free is believed to have superior wound healing properties compared to sheets or materials that include less (e.g., a lower concentration) of amnion.

As is known in the art, amnion graft material can be used to help treat wounds by, for example, providing nutrients for damaged tissue, sealing damaged blood vessels, and/or reducing bleeding. In particular, the amnion material can include nutrients and other materials (e.g., human extracellular matrix components, essential growth factors, and specialized mediating cytokines) that modulate inflation, encourage tissue growth, reduce scar tissue formation, and encourage healing, which makes amnion suitable for wound treatment, as well as for reducing infection and promoting healing. Amnion is also known to provide anti-inflammatory benefits, epithelialization/healing benefits, and scar tissue suppression benefits. The amnion material of the cover device 10 can be provided in a dried or dehydrated state and, as such, can adhere to wetted surfaces, such as patient tissue, without adhesives. In some examples, the amnion sheet is the EvoPatch® implantable patch manufactured by Evologics, LLC of San Antonio, TX, which is a double-layer, chorion-free, dried patch.

The present inventor has recognized that adherence of a protective material or barrier to a surgical site can reduce leakage and bleeding. However, conventional practices for introducing such barrier structures over a wound are often imprecise and/or incomplete. Furthermore, such implanted sheets often detach from biological tissue (e.g., at an intestinal surface) during or after surgery (referred to as either immediate or delayed migration) and/or anastomotic coverage is often incomplete. In order to address such deficiencies, the medical devices disclosed herein are configured to be fixed to the surgical site by, for example, staples, sutures, and/or glue, providing a complete and stable seal over an anastomosis.

In some examples, the surgical methods of the present disclosure can be implemented in open, laparoscopic, and robotic surgeries. For example, actions such as positioning or deployment of the medical device (e.g., the substrate or cover device 10) and/or fixation of the medical device to the surgical site by stapling and/or sutures can be performed using a robotic-assisted surgical system comprising a robotic arm that supports and manipulates a tool, such as a surgical stapler. An exemplary robotic-assisted surgical system that can be used with the medical devices and surgical methods disclosed herein is the Da Vinci™ Robotic-Assisted Surgery system manufactured by Intuitive Surgical, Inc. of Sunnyvale, CA.

### Implantable Cover Device for Anastomosis Procedure

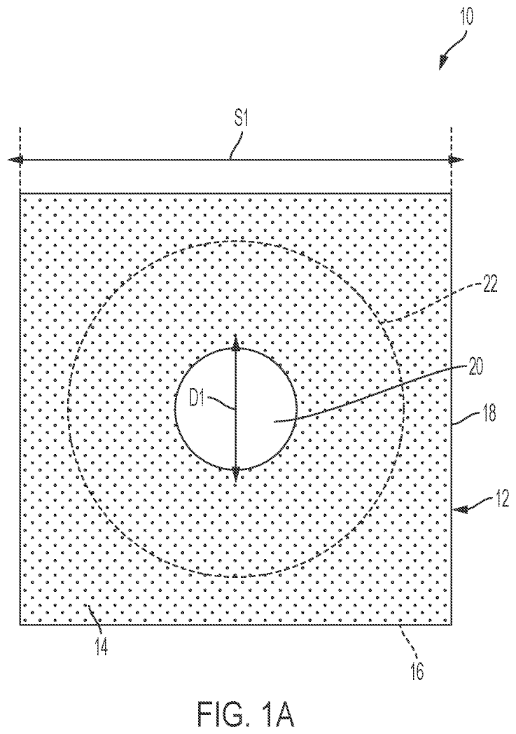

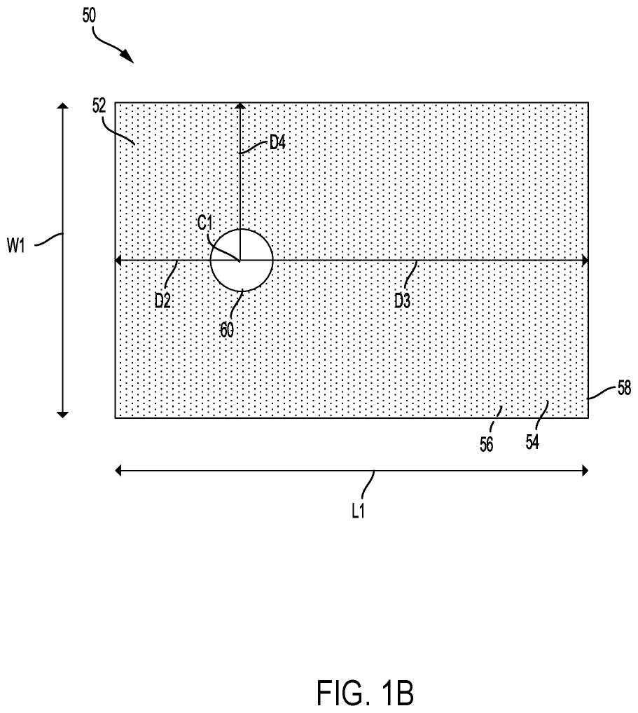

FIG. 1A shows an example of a medical device, referred to herein as an implantable surgical cover device 10, configured to be implanted over tissue of a patient at a surgical site during a surgical procedure, such as an anastomosis surgical procedure. The cover device 10 comprises a substrate, such as a bandage or sheet 12, which includes at least one layer of amnion (also referred to as amnion graft material). As previously described, the amnion sheet can be similar to and/or can include the same materials as the EvoPatch® by Evologics, LLC.

The sheet 12 can include a first or outwardly facing surface 14 configured to face away from the patient tissue, a second or inwardly facing surface 16 configured to contact and/or adhere to patient tissue, and a peripheral edge 18 extending between the outwardly facing surface 14 and the inwardly facing surface 16. In some examples, the outwardly facing surface 14 and the inwardly facing surface 16 of the sheet 12 are identical and are both capable of being attached and/or adhered to patient tissue. In other examples, only the inwardly facing surface 16 comprises dried amnion and is configured to contact the patient tissue, while the outwardly facing surface 14 can be formed from a different material that does not adhere to patient tissue.

The sheet 12 can be any convenient shape and size sufficient for positioning over surfaces, tissues, openings, fenestrations, apertures, or incisions at a surgical site. For example, as shown in FIG. 1A, the sheet 12 can be a square with a side of length S1. The length S1 can be, for example, from about 4 cm to about 8 cm. In other examples, the sheet 12 can be a rectangle, such as a 4 cm by 8 cm rectangular sheet or a 5 cm by 7 cm rectangular sheet.

The cover device 10 also includes an opening or fenestration, such as an annular opening 20 extending through the sheet 12. The annular opening 20 can be positioned on the sheet 12 to guide the surgeon in proper positioning of a surgical tool, such as an anvil of a surgical stapler. For example, as described in further detail in the following description of anastomosis methods, the surgeon may position the cover device 10 on patient tissue and then insert parts of a surgical tool, such as the anvil portion of the surgical stapler, through the annular opening 20 of the cover device 10. As shown in FIG. 1A, the annular opening 20 can be positioned in the center of the sheet 12. The annular opening 20 can be sized and/or configured to permit passage of the anvil of the surgical stapler through the annular opening 20. For example, the annular opening 20 can have a diameter D1 that is slightly larger (e.g., from 1% larger to 10% larger) than an outer diameter of the anvil portion of the surgical stapler. In some examples, the diameter D1 of the annular opening 20 is about 5 mm to about 20 mm. In some examples, the cover device 10 can also include guidelines or markings, such as a circular staple guide 22 showing the surgeon where staples should be deployed. As shown in FIG. 1A, the opening may be an annular opening 20, depicted as a circle. In other examples, while the opening is shown as an annular opening in the various aspects and embodiments described herein the opening, e.g., the annular opening 20, can be any other regular or irregular shape, such as an oval, square, rectangle, or any other convenient and/or useful shape, for example depending on the shape and size of a surgical tool being used for the procedure being performed.

FIG. 1B is a schematic drawing showing another example of an implantable surgical cover device 50, configured to be implanted over tissue of a patient at a surgical site during a surgical procedure, such as an anastomosis surgical procedure. As in previous examples, the cover device 50 comprises a substrate, such as a bandage or sheet 52, which includes at least one layer of amnion (also referred to as amnion graft material). As previously described, the amnion sheet can comprise two amnion layers, such as a double-layer, chorion-free, dried substrate or sheet 52. The sheet 52 can include the same materials as the EvoPatch® by Evologics, LLC.

As in previous examples, the sheet 52 can include a first or outwardly facing surface 54 configured to face away from the patient tissue, a second or inwardly facing surface 56 configured to contact and/or adhere to patient tissue, and a peripheral edge 58 extending between the outwardly facing surface 54 and the inwardly facing surface 56. As in previous examples, the outwardly facing surface 54 and the inwardly facing surface 56 of the sheet 52 can be identical, meaning that both surfaces 54, 56 are capable of being attached and/or adhered to patient tissue.

As shown in FIG. 1B, the sheet 52 is a rectangle having a width W1 and a length L1. For example, as shown in FIG. 1B, the sheet 52 can have a width W1 of about 5 cm and a length L1 of about 7 cm rectangle. In other examples, the sheet 52 can be a 4 cm by 8 cm rectangle. The cover device 50 also includes an annular opening 60 extending through the sheet 52. Unlike in previous examples, which included a centered annular opening, the annular opening 60 in FIG. 1B is offset from the center of the sheet 52. For example, the annular opening 60 can be offset by a distance of about 0.5 cm to 1.5 cm from a center of the sheet in any direction (e.g., either a horizontal or a vertical direction). As shown in FIG. 1B, in one non-limiting example, a center C1 of the annular opening 60 can be a distance D2 of 2.0 cm from one side of the sheet 52 and a distance D3 of 5.0 cm from an opposing side of the sheet 52. As shown in FIG. 1B, the annular opening 60 is spaced an equal distance (e.g., a distance D4 of 2.5 cm) from a top edge and a bottom edge of the sheet 52. However, in other examples, the offset annular opening 60 can be positioned at other locations on the sheet 52 depending on therapeutic requirements of a procedure being performed. For example, the annular opening 60 may be positioned closer to a top edge or a bottom edge of the sheet 52 in some examples.

As described in further detail in connection with the surgical methods of the present disclosure, the offset annular opening 60 is provided to guide the surgeon in positioning an anvil portion of a surgical stapler in a location that is offset from a central axis of lumen(s) of sections or segments of tissue being connected together. In some instances, positioning the surgical stapler at this offset (e.g., spaced apart from the central axis of the tubular section) location allows the surgeon to create an angled end-to-end anastomosis. Also, in some cases, leakage or bleeding of an end-to-end anastomosis is more likely to occur near sides or edges of tissue being stapled together. Therefore, surgeons may wish to position the stapler nearer to such areas to prevent leaks from occurring in such potentially problematic locations. The cover device 50 with the offset annular opening 60 can guide the surgeon in positioning of the surgical stapler proximate to these problematic locations.

As in previous examples, the annular opening 60 can be sized and/or configured to permit passage of the anvil of the surgical stapler through the annular opening 60. For example, the annular opening 60 can have a diameter that is slightly larger than an outer diameter of the anvil portion of the surgical stapler. In some examples, the diameter of the annular opening 60 can be about 5 mm to about 20 mm.

### Surgical Methods for Anastomosis

The implantable cover devices 10, 50 of the present disclosure are configured to be fixed to patient tissue during surgical procedures, particularly in forming an anastomosis for tubular structures of the GI tract. Such procedures generally involve forming either an end-to-end anastomosis or a side-to-side anastomosis between the tubular structures (e.g., an intestine or bowel) of the GI tract. The anastomosis techniques described herein can also be transferable to other intra-abdominal procedures involving, for example, intra or extra peritoneal anastomosis, bariatrics, complex appendectomy, small bowel resections, and similar procedures.

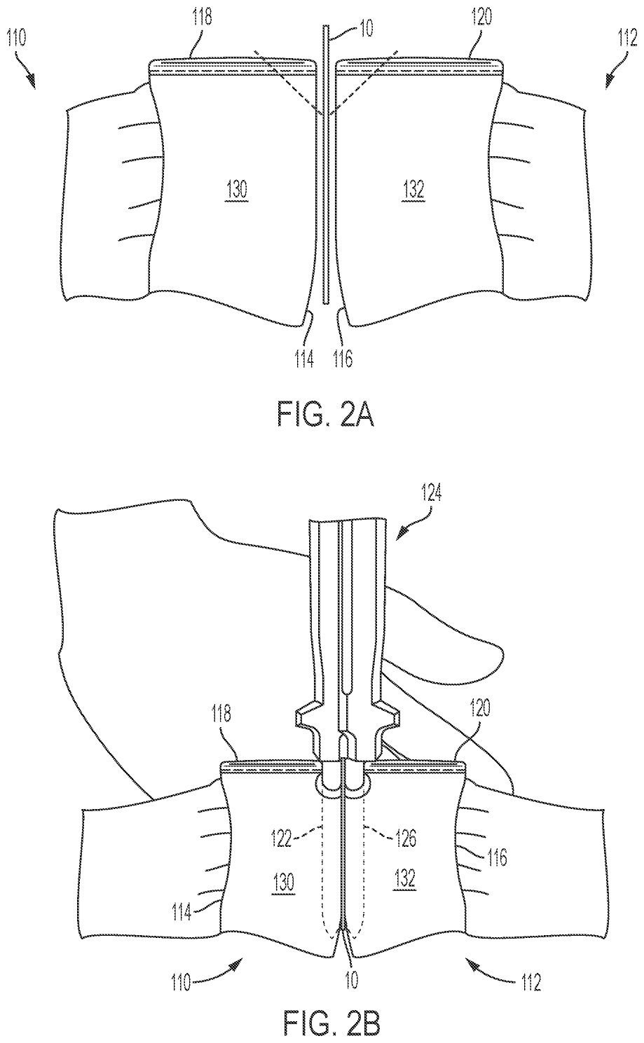

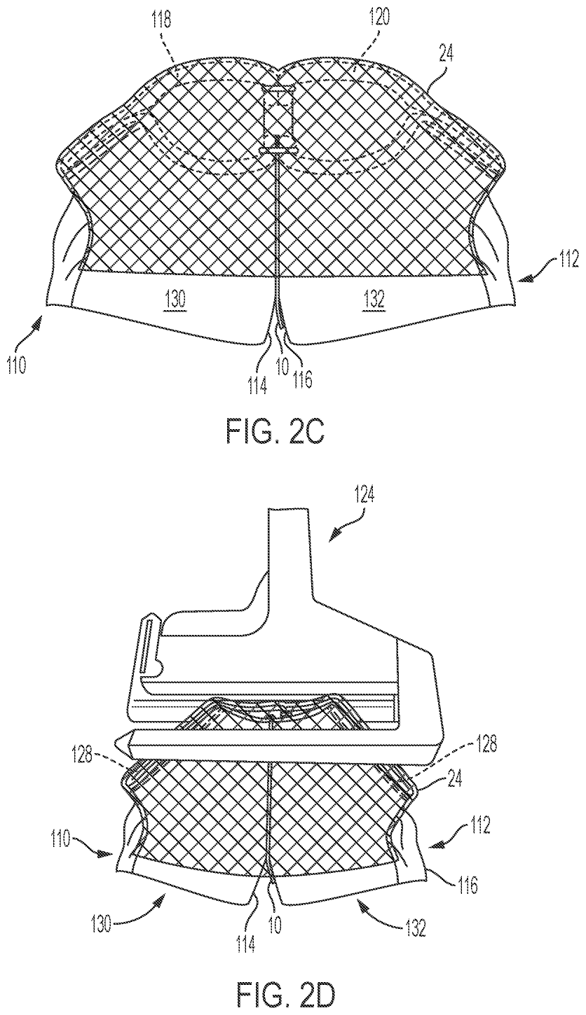

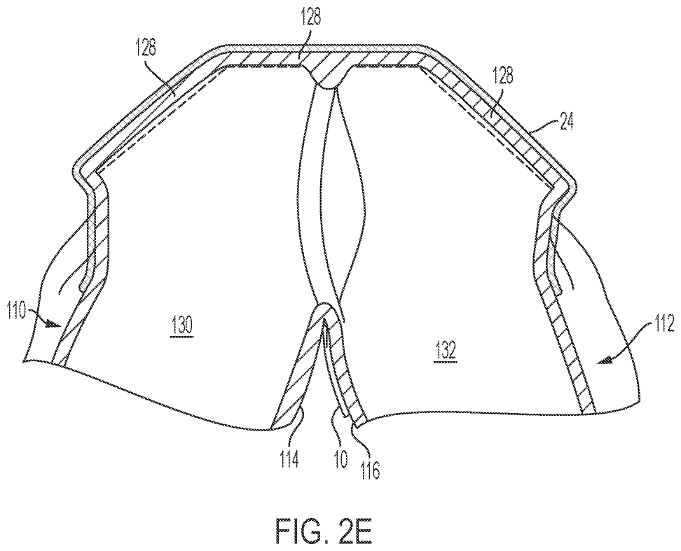

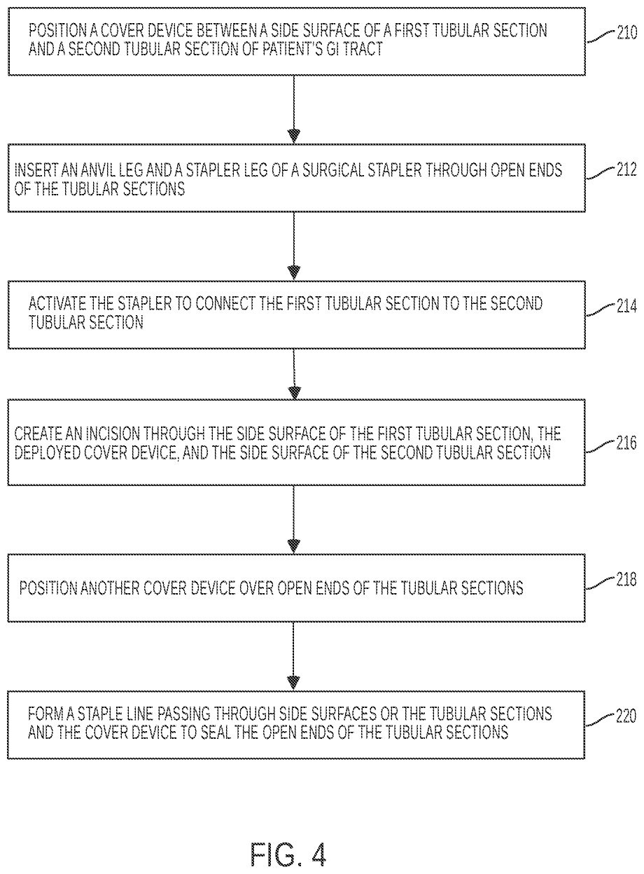

FIGS. 2A-2E are schematic drawings showing steps performed by a surgeon in forming a side-to-side anastomosis, which includes positioning a first cover device 10 between side surfaces 114, 116 of the tubular structures 110, 112 and a second cover device 24 over open ends 118, 120 of the tubular structures 110, 112. After the cover devices 10, 24 are in place, the tubular structures 110, 112 can be connected together by stapling or suturing, such that at least some staples or sutures pass through the cover devices 10, 24. FIG. 4 is a flow chart showing the steps of the side-to-side anastomosis surgical method.

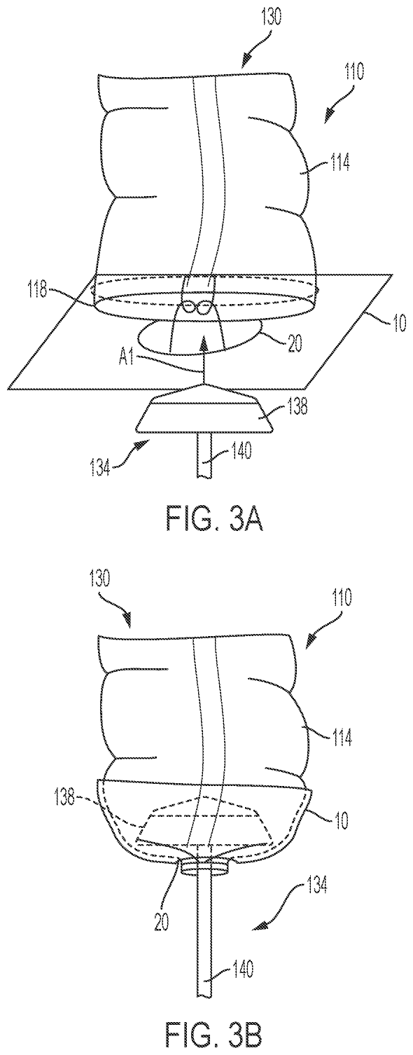

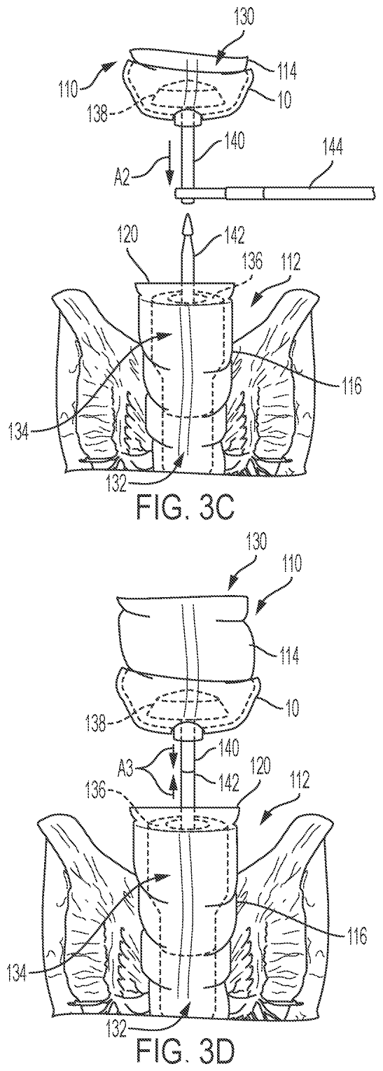

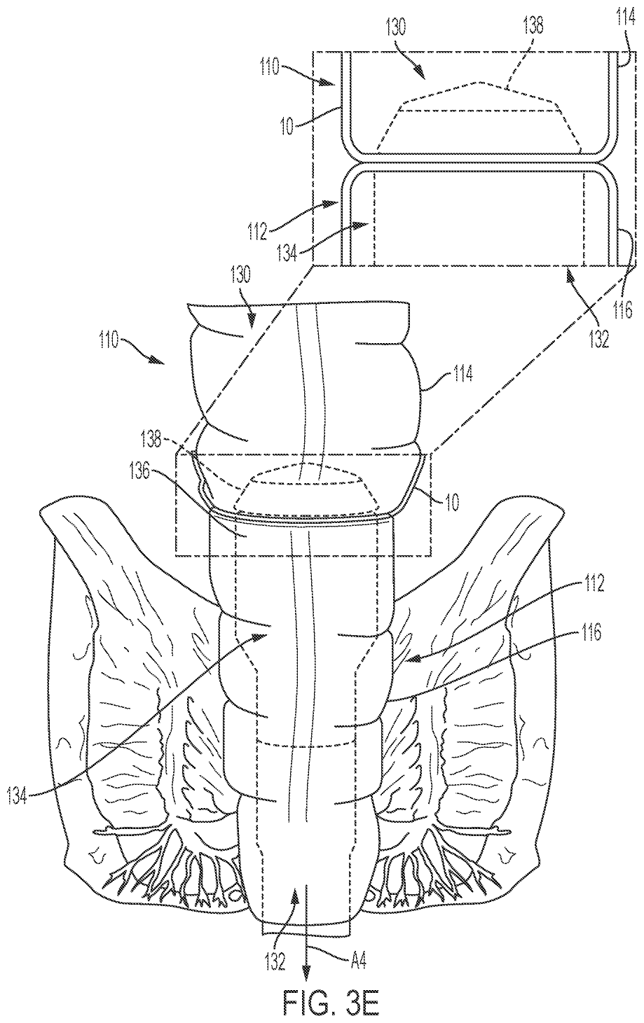

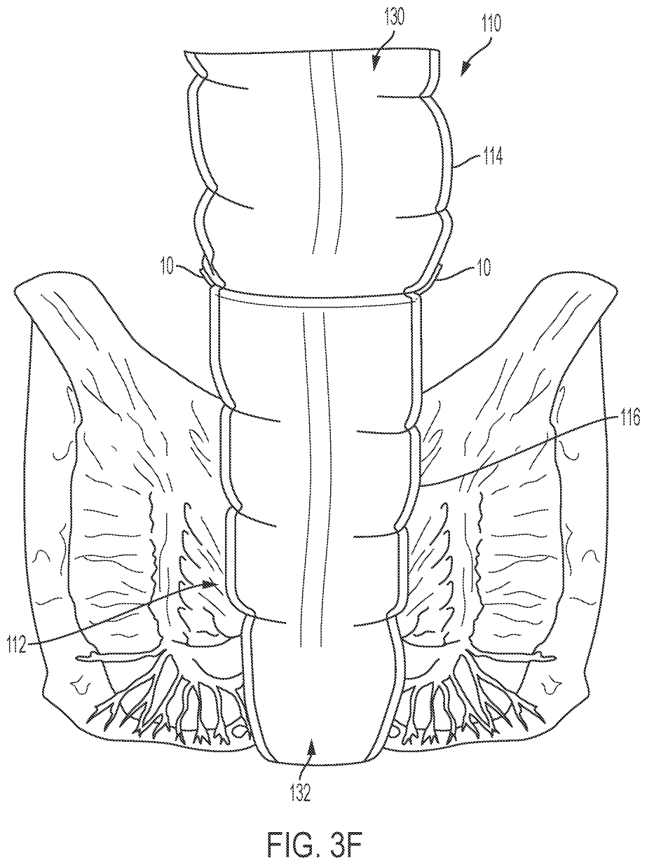

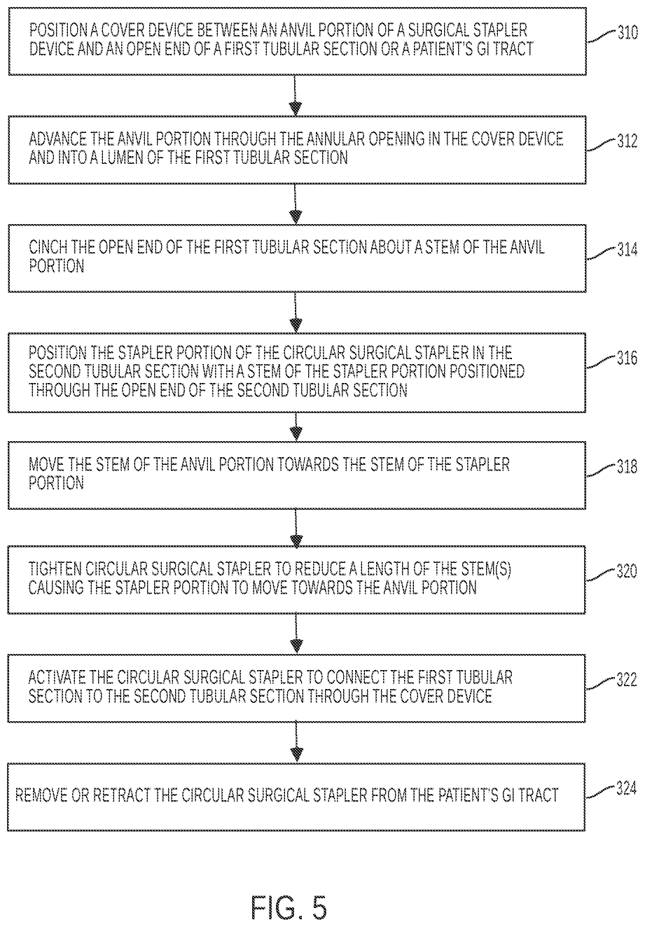

FIGS. 3A-3F are schematic drawings showing steps performed by the surgeon in forming an end-to-end anastomosis, which include deploying or positioning the cover device 10 between open ends 118, 120 of tubular structures 110, 112 of the GI tract prior to connecting the open ends 118, 120 of the tubular structures 110, 112 together. Once the cover device 10 is in place, the tubular structures 110, 112 can be connected together by stapling or suturing, such that at least some staples or sutures pass through the cover device 10. FIG. 5 is a flow chart showing steps performed for the end-to-end anastomosis method.

As shown in FIG. 4, the method for forming the side-to-side anastomosis includes, at step 210, positioning a cover device 10 (e.g., any of the previously described cover devices formed from one or more amnion layers) between a side surface 114 of the first tubular structure, segment, or section 110 of patient tissue (e.g., an intestine or bowel of a patient's GI tract) and a side surface 116 of a second tubular section 112 of patient tissue. FIG. 2A is a schematic drawing showing the cover device 10 between the tubular sections 110, 112 of patient tissue. The cover device used for the side-to-side anastomosis can be either a square device 10, as shown in FIG. 1A, or a rectangular device 50 shown in FIG. 1B. For the side-to-side anastomosis, the annular opening 20, 60 of the cover device 10, 50 can be either centered on the sheet 12, 52 or offset. Also, if greater coverage is needed, the cover device 10, 50 can be positioned so that a staple line passes through a diagonal of the cover device 10, 50 (e.g., from a top left edge to a bottom right edge of the cover device 10, 50). Positioning the cover device 10, 50 in this manner allows the cover device 10, 50 to seal against a longer staple line than could be achieved in other orientations.

At step 212, the method further includes inserting an anvil leg 122 of a surgical stapler 124 through an open end 118 of one of the tubular sections 110, 112 and a staple leg 126 of the surgical stapler 124 through the open end 120 of the second tubular section 112, as shown in FIG. 2B. Surgical staplers 124 including a staple leg 126 and anvil leg 122 for deploying lines of staples are known in the art and commercially available from manufactures including Ethicon US, LLC and Medtronic plc. The surgical stapler 124 can also be a tool configured to be manipulated by a robotic-assisted surgery system. An exemplary surgical stapler 124, which can be used for this surgical method, is disclosed in U.S. Pat. No. 8,453,908 entitled “Surgical stapling instrument with improved firing trigger arrangement”. At step 214, the method further includes connecting the first tubular section 110 to the second tubular section 112 by activating the surgical stapler 124 causing staples to pass from the staple portion 126 of the surgical stapler 124, through the side surfaces 114, 116 of the first tubular section 110 and the second tubular section 112 and through the cover device 10. Passing the staples through the cover device 10 fixes the cover device 10 between the tubular sections 110, 112, preventing the cover device 10 from detaching from tissue or migrating during or following completion of the surgical procedure.

At step 216, the method further includes forming an incision through the side surface 114 of the first tubular section 110, the deployed cover device 10, and the side surface 116 of the second tubular section 112. The incision can be made using a scalpel or similar surgical cutting tool. In some examples, the incision can be formed by a surgical tool manipulated by the robotic-assisted surgery system.

At step 218, the method further includes positioning a second cover device 24, such as another amnion patch or sheet, over open ends 118, 120 of the tubular sections 110, 112, as shown in FIG. 2C. The second or another cover device 24 can be either square (as shown in FIG. 1A) or rectangular (as shown in FIG. 1B) in shape. Further, the annular opening 20, 60 of the cover device 24 can be either centered or offset. At step 220, the method further includes forming a staple line 128 passing through the side surface 114 of the first tubular section 110, the side surface 116 of the second tubular section 112, and the cover device 24. As shown in FIG. 2D, the staple line 128 seals the open ends 118, 120 of the tubular sections 110, 112. Sealing the open ends 118, 120 of the tubular sections 110, 112 forms a continuous lumen extending from a lumen 130 of the first tubular section 110, through the incision, and to a lumen 132 of the second tubular section 112. After forming the staple line 128, any excess tissue above the staple line 128 can be excised. Also, any excess portions of the cover device 24 can be folded over the staple line 128 and/or excised tissue to improve fixation and/or enhance healing of incisions and/or damaged tissue. Desirably, due to the positioning of the cover device 24, there is no active bleeding through or around the staple line 128. FIG. 2E is a schematic drawing showing the completed side-to-side anastomosis with the continuous lumen extending between the first tubular section 110 and the second tubular section 112.

FIG. 5 is a flow chart showing steps for forming the end-to-end anastomosis, which includes positioning the cover device 10 over an open end 118 of one of the tubular sections 110, 112 being joined together. In the following description, the cover device 10 is described as being positioned proximate to an anvil portion of a surgical stapler. However, when forming the end-to-end anastomosis, the cover device 10 can also be positioned proximate to a staple portion of the surgical stapler with similar efficiency. FIGS. 3A-3F are schematic drawings showing steps performed during the surgical procedure. The procedure uses a surgical stapler device, particularly an intraluminal circular surgical stapler device 134, comprising a circular staple portion 136 and anvil portion 138. The circular surgical stapler device 134 is configured to provide staples in a circular pattern in a lumen of a tubular structure. Exemplary circular surgical stapler devices 134, which can be used with the methods disclosed herein are available from Ethicon LLC, Medtronic plc, and others. An exemplary circular stapler device 134, which can be used for the surgical methods disclosed herein, is described in U.S. Pat. No. 9,597,082 entitled “Method and apparatus for sealing end-to-end anastomosis”.

With reference to FIG. 5, the method includes, at step 310, positioning a cover device 10, such as a device comprising an amnion sheet, between the anvil portion 138 of the circular surgical stapler device 134 and an open end 118 of a first tubular portion, segment, or section 110 of, for example, the patient's GI tract. The cover device 10 can be a square cover device 10 with an annular opening 20 in the center of the cover device 10. The annular opening 20 of the cover device 10 can be aligned with central axes of lumens 130, 132 of the tubular section 110. In other examples, the cover device 50 can include the offset annular opening 60 shown in FIG. 1B, which is configured to be offset from the central axis of the lumens 130, 132 of the tubular sections 110, 112.

As shown in FIG. 3A, at step 312, the anvil portion 138 can be advanced, in a direction of arrow A1, through the annular opening 20 in the cover device 10 and into a lumen of the first tubular section 110. As previously described, the cover device 10 can also be positioned proximate to the staple portion 136 with similar efficiency.

At step 314, the method can further include cinching the open end 118 of the first tubular section 110 about a stem 140 of the anvil portion 138 by, for example, tightening sutures positioned around the open end 118 of the first tubular section 110. The open end 118 of the first tubular section 110 is shown in this tightened or cinched position in FIG. 3B.

At step 316, the method further includes positioning the staple portion 136 of the circular surgical stapler 134 in the second tubular section 112, such that a stem 142 of the staple portion 136 passes through the open end 120 of the second tubular section 112. The staple portion 136 is shown positioned in the second tubular section 112 in FIG. 3C.

At step 318, as shown in FIG. 3C, the method further includes moving the stem 140 of the anvil portion 138 towards the stem 142 of the staple portion 136, as shown by arrows A2, using, for example, a clamp 144 or similar tool. The stem 140 of the anvil portion 138 is shown engaged to the stem 142 of the staple portion 136 in FIG. 3D.

At step 320, once the stems 140, 142 of the anvil portion 138 and the staple portion 136 are connected together, the method includes tightening the circular surgical stapler 134, which reduces a length of the stem(s) 140, 142 and causes the staple portion 136 to move towards the anvil portion 138, as shown by arrows A3 in FIG. 3D.

At step 322, once the staple portion 136 and the anvil portion 138 are fully tightened together as shown in FIG. 3E, the method further includes activating the circular surgical stapler 134 to project staples from the staple portion 136 through the tubular sections 110, 112 and the cover device 10, thereby connecting the first tubular section 110 to the second tubular section 112. As previously described, the circular surgical stapler 134 is configured to deploy staples in a circular pattern. The deployed staples can extend about the lumens 130, 132 of the first tubular section 110 and/or the second tubular section 112, thereby forming a single continuous lumen passing through an opening or space enclosed by the circle of staples. In other examples, such as when using the cover device 50 with the offset annular opening 60, the deployed staples can be offset from the central axis of the lumen 130, 132. For example, as previously described, deploying the staples offset from the central axis of the lumen 130, 132 may prevent leakage from certain problematic locations near edges or sides of tubular sections 110, 112 being connected together by the end-to-end anastomosis.

At step 324, after the staples are deployed through the tubular sections 110, 112 and the cover device 10, the circular surgical stapler 134 can be removed or retracted from the patient's GI tract by moving the anvil portion 138 and staple portion 136 of the surgical stapler away from the formed anastomosis in a direction of arrow A4 (shown in FIG. 3E). FIG. 3F shows the formed end-to-end anastomosis after removal of the circular surgical stapler 134 from the lumens 130, 132 of the tubular sections 110, 112. As shown in FIG. 3F, once the circular surgical stapler 134 is removed, the lumens 130, 132 form a continuous and unobstructed lumen extending between portions of the GI tract of the patient.

### EXAMPLES

In order to show that the cover devices 10 comprising amnion disclosed herein provide an effective seal for reducing leaks in anastomosis procedures, results for surgical procedures performed at a medical facility over a three month period were documented. Specifically, the study included patients who underwent abdominal surgery in which a primary bowel anastomosis was created. Patients were treated either “without amnion” meaning that an amnion sheet or cover device was not positioned over the surgical site or “with amnion” meaning that a cover device or amnion sheet was fixed to the surgical site by a surgical stapler during the procedure. The “without amnion” and “with amnion” groups of patients were compared for anastomotic leak rates, readmissions, re-explorations, and other complications.

### Surgical Procedures without Amnion

Eighty-four surgical patients (50 female patients (59.5%) and 34 male patients (40.4%)), underwent an abdominal surgical procedure including a primary intestinal anastomosis creation without use of an amnion sheet or cover device. 72% of these procedures were performed with either a robotic or laparoscopic approach (66% robotic, 6% laparoscopic). 28% of the procedures were either planned open surgical procedures or converted to open procedures. The median patient age at the time of operation was 64.4 years old.

Of the 84 cases studied: 31 patients' pre-operative diagnosis was colon/rectal cancer (36.9%); 32 patients' pre-operative diagnosis was diverticulitis (38%), seven cases (22%) of which were complicated by perforation prior to exploration; seven patients' preoperative diagnosis was defined as “ostomy takedown” (8.3%); seven patients' preoperative diagnosis was small intestinal obstruction (7.1%); two cases with free perforation prior to arrival at the operating room; one patient with causation secondary to volvulus; and one patient with causation secondary to intussusception. Less than 1% of cases had the preoperative diagnosis of small intestinal perforation prior to operation and/or ischemia prior to exploration. All 84 surgical patients studied received close post-operative monitoring with inpatient hospitalization or observation for at least 12 hours post-operation.

Of the patients studied, 10 patients were readmitted (admission prior to 30 days post-discharge) to a hospital facility (12%). Of those patients, the readmitting diagnosis of primary anastomotic failure, defined as bowel content spillage into the abdominal cavity at any time post-procedure, was 5 patients meaning that the overall leak rate was 5.7% (5/84). The overall survival rate of those five patients was 100%. The average hospital stay for those patients with an anastomotic leak was >7 days. 4 of the 5 patients required reoperation. Three patients required 2 additional operations. One patient developed a colo-vaginal fistula and eventually required 3 additional surgeries.

### Surgical Procedures with Amnion

79 surgical patients (53 female patients (67%) and 26 male patients (32.9%)), underwent an abdominal surgical procedure including a primary intestinal anastomosis creation. 75% of the surgical procedures were robotic, 24% were either planned open surgical procedures or resulted in open procedures, and 1% were laparoscopically performed. The median patient age at the time of operation was 65.2 years old.

Of the 79 cases studied: 36 patients' pre-operative diagnosis was colon/rectal cancer (45.5%); 20 patients' pre-operative diagnosis was diverticulitis (23.3%), three cases (13%) of which were complicated by perforation prior to exploration; seven patients' pre-operative diagnosis was defined as “ostomy takedown” (8.8%); three patients' pre-operative diagnosis was small intestinal obstruction (3.7%); and one patient with causation secondary to volvulus. All 79 surgical patients studied received close post-operative monitoring with inpatient hospitalization or observation for at least twelve hours post-operation. In each of the 79 procedures, a cover device or amnion sheet, as disclosed herein, was positioned within the surgical site. The cover device 10 was a 6 cm square device. The surgical procedures involved forming a side-to-side anastomosis according to the method of FIG. 4 and/or an end-to-end anastomosis according to the method of FIG. 5. As previously described, during the procedures, staples passed through the cover device 10 to fix the cover device 10 to the surgical site preventing migration of the cover device 10.

No leaks were detected or reported in any of these seventy-nine (79) intestinal anastomoses procedures using the amnion cover device 10, meaning that the leak rate was 0% (0/79).

In view of the results, the present inventor believes that implementation of a cover device comprising amnion in primary intestinal anastomoses shows promising results in decreasing anastomotic leaks and associated complications. In particular, the inventor believes that the study shows that the use of the cover device or amnion sheet appears to significantly reduce the number of anastomotic leaks in intestinal surgery and may therefore reduce the prolonged hospital length of stay and/or the need for readmission, suggesting benefits to both patient outcomes and hospital cost-effectiveness. Follow-on work with cover devices 10, 50 comprising amnion of the present disclosure provided similar results evidencing the effectiveness of the cover device 10, 50 and amnion for improving safety and reducing risks associated with intestinal anastomosis procedures.

Although the disclosure has been described in detail for the purpose of illustration based on what are currently considered to be the most practical and preferred aspects, it is to be understood that such detail is solely for that purpose and that the disclosure is not limited to the disclosed aspects, but, on the contrary, is intended to cover modifications and equivalent arrangements. For example, it is to be understood that the present disclosure contemplates that, to the extent possible, one or more features of any aspect can be combined with one or more features of any other aspect.

What is claimed is:

1. An implantable surgical cover device configured to be implanted over tissue of a patient at a surgical site during a surgical stapling procedure, the cover device comprising:
at least one sheet comprising at least one layer of amnion, wherein the at least one sheet comprises a first surface, a second surface, and a peripheral edge extending between the first surface and the second surface; and
at least one opening extending through the at least one sheet provided to guide a user for proper positioning of a surgical stapler during the surgical stapling procedure.

2. The cover device of claim 1, wherein the at least one layer of amnion comprises a dried sheet of chorion-free amnion.

3. The cover device of claim 1, wherein the at least one sheet comprises a one ply, two ply, three ply, or four ply amnion sheet.

4. The cover device of claim 1, wherein the at least one sheet is a dried sheet comprising at least two layers of chorion-free amnion.

5. The cover device of claim 1, wherein the at least one sheet is a square shape with an edge of from about 4 cm to about 8 cm in length, wherein the at least one opening has a diameter of from about 5 mm to about 20 mm, and wherein the opening is optionally an annular opening.

6. The cover device of claim 1, wherein the at least one opening is sized to permit an anvil portion of a circular surgical stapler to pass through the at least one opening, and wherein the opening is optionally an annular opening.

7. The cover device of claim 1, wherein the at least one sheet is rectangular and the at least one opening is offset from a center of the at least one sheet by at least 0.5 cm in at least one direction.

8. A surgical method comprising:
positioning at least one sheet comprising amnion between a first tubular section of patient tissue and a second tubular section of the patient tissue; and
connecting the first tubular section to the second tubular section by at least one of stapling or sutures, such that at least some staples or sutures pass through the at least one sheet, thereby connecting the first tubular section to the second tubular section, with the at least one sheet comprising amnion between the first tubular section and the second tubular section.

9. The method of claim 8, wherein the first tubular section defines a first lumen and the second tubular section defines a second lumen, and
wherein connecting the first tubular section to the second tubular section places the first lumen in fluid communication with the second lumen.

10. The method of claim 9, wherein the at least one sheet comprises an opening extending through surfaces of the at least one sheet,
wherein positioning the at least one sheet comprises positioning the at least one sheet over the first tubular section such that the opening is aligned with the first lumen, and wherein the opening is optionally an annular opening.

11. The method of claim 10, wherein connecting the first tubular section to the second tubular section comprises:
positioning an anvil portion of a circular surgical stapler in the first lumen of the first tubular section, such that a stem of the anvil portion extends through the opening of the at least one sheet, wherein the opening is optionally an annular opening;
positioning a staple portion of the circular surgical stapler in the second lumen of the second tubular section;
tightening the circular surgical stapler, thereby causing the staple portion to move towards the anvil portion; and the first tubular section to move towards the second tubular section and
activating the circular surgical stapler to project a plurality of staples from the staple portion through the first tubular section, the at least one sheet, and the second tubular section, thereby connecting the first tubular section to the second tubular section.

12. The method of claim 11, wherein the circular surgical stapler comprises a gastrointestinal stapler.

13. The method of claim 12, wherein the opening comprises a diameter that is slightly larger than a diameter of the anvil portion, thereby allowing the anvil portion to pass through the opening without contacting the at least one sheet, and wherein the opening is optionally an annular opening.

14. The method of claim 8, wherein a surgical procedure of the surgical method is an end-to-end anastomosis in which an end of the first tubular section is connected to an end of the second tubular section.

15. The method of claim 8, wherein a surgical procedure of the surgical method is a side-to-end anastomosis, in which an end of the first tubular section is connected over an opening in a sidewall of the second tubular section.

16. The method of claim 8, wherein a surgical procedure of the surgical method is a side-to-side anastomosis.

17. The method of claim 16, wherein positioning the at least one sheet between the first tubular section and the second tubular section comprises positioning a first at least one sheet between a side surface of the first tubular section and a side surface of the second tubular section.

18. The method of claim 17, wherein connecting the first tubular section to the second tubular section comprises stapling the side surface of the first tubular section to the side surface of the second tubular section through the first at least one sheet,
the method further comprising forming an incision through the side surface of the first tubular section, the first at least one sheet, and the side surface of the second tubular section, thereby forming a passage between a lumen defined by the first tubular section and a lumen of the second tubular section.

19. The method of claim 17, further comprising:
positioning a second at least one sheet comprising amnion over open ends of the first tubular section and the second tubular section; and
forming a staple line through the first tubular section and the second tubular section and the second at least one sheet comprising amnion, thereby sealing the open ends of the first tubular section and second tubular section and forming an enclosed lumen defined by the first tubular section and the second tubular section.

20. The method of claim 8, wherein a surgical procedure of the surgical method is a robotic-assisted surgical procedure, and wherein connecting the first tubular section to the second tubular section comprises automatically stapling the first tubular section to the second tubular section using a surgical robot system comprising at least one arm that moves and actuates a surgical stapler.

21. The method of claim 8, wherein a surgical procedure of the surgical method comprises an anastomosis procedure, and wherein the first tubular section and the second tubular section comprise sections of a gastrointestinal (GI) tract of a patient.

22. The method of claim 21, wherein the sections of the GI tract comprise at least one of a bowel or intestine of the patient.

23. A surgical method for forming a side-by-side anastomosis, comprising:
positioning at least one sheet comprising amnion between a side surface of a first tubular section of patient tissue and a side surface of a second tubular section of the patient tissue;
inserting an anvil leg of a surgical stapler through an open end of one of the first tubular section or the second tubular section and inserting a staple leg of the surgical stapler through an open end of the other of the first tubular section or the second tubular section;
connecting the first tubular section to the second tubular section by activating the surgical stapler causing staples to pass from the staple leg, through the side surfaces of the first tubular section and the second tubular section, and through the at least one sheet;
positioning another at least one sheet comprising amnion over the open end of the first tubular section and the second tubular section; and
forming a staple line passing through the side surface of the first tubular section, the side surface of the second tubular section, and the another at least one sheet, wherein the staple line seals the open end of the first tubular section and the open end of the second tubular section forming a continuous lumen extending from the first tubular section to the second tubular section.

24. The method of claim 23, wherein a surgical procedure of the surgical method comprises an anastomosis procedure, and wherein the first tubular section and the second tubular section comprise sections of a gastrointestinal (GI) tract of a patient.

25. The method of claim 24, wherein the sections of the GI tract comprise at least one of a bowel or intestine of the patient.

26. The method of claim 23, further comprising forming an incision through the side surface of the first tubular section, the at least one sheet, and the side surface of the second tubular section, thereby forming the continuous lumen defined by the first tubular section and the second tubular section.

27. The method of claim 23, wherein a surgical procedure of the surgical method is a robotic-assisted surgical procedure, and wherein connecting the first tubular section to the second tubular section by activating the surgical stapler comprises automatically stapling the first tubular section to the second tubular section using a surgical robot system comprising at least one arm that moves and actuates the surgical stapler.
